(19) United States
(12) Patent Application Publication
(10) Pub. No.: US 2012/0316129 A1
(43) Pub. Date: Dec. 13, 2012

(54) LIQUID DRESSING CONTAINING CHITOSAN DERIVATIVE AND PREPARATION METHOD THEREOF

(76) Inventors: Yuqiang LI, Qingdao City (CN); Shixue SUN, Qingdao City (CN)

(21) Appl. No.: 13/493,698

(22) Filed: Jun. 11, 2012

(30) Foreign Application Priority Data
Jun. 10, 2011 (CN) 201110155717

(51) Int. Cl. A61K 31/722; A61P 17/02

(52) U.S. Cl. 514/54

(57) ABSTRACT

The invention relates to a liquid dressing containing a chitosan derivative, comprising the following components in percentage by weight: 1-5% of CMCS (carboxymethyl chitosan), 0.1-0.5% of collagen albumen powder, 10-15% of humectant, 0.2-2% of antibacterial agent, 2-10% of penetration enhancers and the balance of deionized water. The liquid dressing is obtained by mixing the components in sequence, stirring evenly and bulking. The liquid dressing has the advantage of good biocompatibility, good antimicrobial, antiphlogistic and hemostatic effects and high biological safety, and is convenient to use.

### FIELD OF THE INVENTION

The invention belongs to the technical field of medical appliances, which particularly relates to liquid dressing including chitosan derivative.

### BACKGROUND OF THE INVENTION

As the presentation of moist wound healing theory, various novel dressing is arisen. According to incomplete statistics, currently more than 2400 types of wound dressing exist on the market [Krasner D L, Rodeheaver G T, Sibbald R G, et al chronic wound care [M]. 4th ed Malvem, PA:HMP Communications, 2007:249]. How to select the safest, the most economical and effective dressing according to the wound condition and economic capacity of patients is the problem which is offers faced by medical care personnel in clinical working. Scholars at home and abroad do a large amount of researches on the selection and application of dressing, but consensus is not formed presently. The clinical effective research of dressing at home and abroad and relevant guide are referred, and the characteristics of various products and the clinical application conditions are stated, so as to be convenient for providing reference for reasonale clinical wound dressing selection.

Presently clinical medical care personnel at home and abroad has do a large amount of researches on wound dressing cost benefit, but the researches related to the sorts of dressing are incomplete, part of the researches lack effective comparison or comparison group processing methods are not explained, and comprehensive and effective evidence-based reference can not be provided. Therefore, more cost benefit researches of random comparison is required to be performed, to provide reference for the selection of clinical wound dressing.

Ideal dressing should have the following functions: preventing excessive loss of moisture and body fluid, resisting bacterial invasion and preventing infection, proper wounded surface adhering but not adhering to the wounded surface to avoid the secondary injury caused by the changing of dressing band, moisture penetration, air permeability, and ensuring that the wounded surface is in moist environment without accumulated liquid, and good biological compatibility, and optimally promoting the healing of wound. Although biological dressing is rapidly developed, [Courtenay M. Choosing wound dressings [J]. Nursing Times, 1998, 94 (9): 462 48.], no dressing can achieve the requirement of ideal dressing, and all types of dressing have disadvantages that can not be overcome.

### SUMMARY OF THE INVENTION

In order to solve the technical problem, the invention provides liquid dressing including chitosan derivative.

The invention also provides a preparation method of the liquid dressing including chitosan derivative.

The liquid dressing including chitosan derivative comprises the following components by weight percentage: 1 to 5 percent of carboxymethyl chitosan, 0.1 to 0.5 percent of collagen powder, 10 to 15 percent of moisturizing agent, 0.2 to 2 percent of antimicrobial agent, 2 to 10 percent of transdermal enhancer, and the surplus for deionized water.

Optimally the moisturizing agent adopts propanetriol, sorbierite or carbowax (4000-6000).

Optimally the antimicrobial agent adopts triclosan or R-polysaccharide, wherein the weight percentage of the triclosan is 0.2 to 0.3 percent optimally, and the weight percentage of the R-polysaccharide is 0.5 to 2 percent optimally.

Optimally the transdermal enhancer adopts laurocapram, menthol or diethylene glycol-ethyl ether, wherein the weight percentage of the laurocapram is 5 to 10 percent optimally, the weight percentage of the menthol is 5 to 10 percent optimally, and the weight percentage of the diethylene glycol-ethyl ether is 2 to 4 percent optimally.

In one optimal technical scheme, the liquid dressing comprises the following components by weight percentage: 1 to 5 percent of carboxymethyl chitosan, 0.1 to 0.5 percent of collagen powder, 10 to 15 percent of propanetriol, 0.2 to 0.3 percent of triclosan, 5 to 10 percent of laurocapram, and the surplus for deionized water.

The pH value of the liquid dressing including chitosan derivative is between 6.0 to 8.0, and is 6.5 to 7.0 optimally.

In the liquid dressing including chitosan derivative, the carboxymethyl chitosan adopts O-site substitution, or N-site substitution, or O-site substitution and N-site substitution simultaneously, the molecular weight is within 500 thousands to 3 millions, and the substitution value is within 0.5 to 3.

The structural formula of chitosan is as follows:

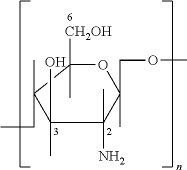

Two hydroxy groups (C₃ and C₆) and one amino group (—NH₂) exist in the molecular structure. Theoretically carboxymethyl can be introduced to the three groups, but positions and electronegativity of the three groups are different, so that the reactive activities of the three groups are different. The electronegativity of oxygen is larger than that of nitrogen, so that the nucleophilic reaction of —OH base is larger than that of —NH₂ base; in C₃ and C₆, the reaction speed of primary alcohol group (C₆) is larger than that of secondary alcohol group (C₃), hydrogen atom on the secondary alcohol can form hydrogen bond with non-shared electron pair on —NH₂, so that hydrogen on C₃—OH is not easy to move off, the carboxy methylation reaction is mainly performed on the primary alcohol group C₆ of chitosan and can also be performed on the amino group, namely the product produced is O-carboxymethyl chitosan or N-carboxymethyl chitosan. The structural formula is as follows:

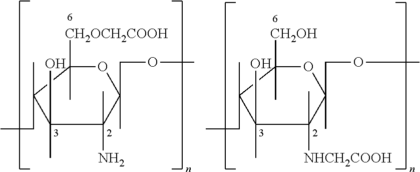

O-carboxymethyl chitosan N-carboxymethyl chitosan

The product mass parameters of the carboxymethyl chitosan are as follows:

Property: off-white powdery solid

Dissolvability: dissolved in deionized water

pH: 6.8

Degree of viscosity (1%, 20° C.), mPa·s: 23.0

Degree of substitution (%): 95.5.

The preparation method of the liquid dressing including chitosan derivative comprises the following steps:

a. weighing carboxymethyl chitosan to be added into the deionized water to be mixed till being completely dissolved, so as to form liquid A for reservation;

b. weighing collagen powder to be added into the deionized water to be mixed till being completely dissolved, so as to form liquid B for reservation;

c. sequentially weighing the transdermal enhancer and the antimicrobial agent to be dissolved in the moisturizing agent in sequence to be uniformly mixed, so as to form liquid C;

d. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;

e. filling.

Optimally preparation method of the liquid dressing including chitosan derivative comprises the following steps:

a. weighing carboxymethyl chitosan to be added into the deionized water to be mixed till being completely dissolved, so as to form liquid A for reservation;

b. weighing collagen powder to be added into the deionized water to be mixed till being completely dissolved, so as to form liquid B for reservation;

c. sequentially weighing the laurocapram and the triclosan to be dissolved in the propanetriol in sequence to be uniformly mixed, so as to form liquid C;

d. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;

e. filling.

The liquid dressing has the functions of antibiosis, sterilization, antiphlogosis, wound healing promoting, scar reducing, wounded surface healing promoting, exudative tissue fluid reducing, accretion preventing, infection preventing and the like.

The liquid dressing gel takes carboxymethyl chitosan and protein as the main functional material, and proper antibiotic substances are added, wherein the water solubility of N, O-carboxymethyl chitosan is good and the transparency is high, the degree of dissociation in water is not affected by pH value, the wetting action of liquid dressing is similar to that hyaluronic acid, and the liquid dressing can form membrane, can form membrane on the ulcerative surface to achieve the purpose of treatment, and is applicable to the practical application of the medicine industry.

Collagen is extracellular matrix and easy to be absorbed, can provide nutrition for lesion sites, and provide carriers for regenerative cells on the wounded surface to promote the adherence and distribution of epithelial cells; by depending on the effect of the matrix, the collagen has drugs slow-release, can carry substance to be slowly released, to ensure that the antibacterial effect is more lasting, so that the wounded surface can maintain good growing environment, the collagen is beneficial to the wound healing, and good treatment effect is achieved. In addition, collagen has auxiliary treatment effect on the formation of scar after laser and photon treatment, can improve the surgical effect and reduce pigmentation, by compounding collagen and carboxymethyl chitosan, the degradation time of collagen can be prolonged, and the anti-infectious performance can be improved.

Triclosan assists the sterilization effect of carboxymethyl chitosan, and can rapidly kill bacteria, eubacteria and virus. The dressing is applied to the burn bounded area, and has the functions of antibiosis, sterilization, antiphlogosis, wound healing promoting, scar reducing, wounded surface healing promoting, exudative tissue fluid reducing, accretion preventing, infection preventing and the like.

The liquid dressing has the following advantages of firstly, good biological compatibility, secondly, good antibiosis, antiphlogosis and hemostasis effects, thirdly, high biological safety; fourthly, good product quality, proper moderate cost, convenient use, and high market competitiveness.

### MODE OF CARRYING OUT THE INVENTION

Embodiments bellows are used for illustrating the invention, but not limited the range of the invention.

### Embodiment I

Components are taken by weight percentage as follows:

By taking 1000 g liquid dressing as reference, 1 percent of carboxymethyl chitosan, 0.5 percent of collagen powder, 10 percent of propanetriol, 0.2 percent of triclosan, 5 percent of laurocapram are taken, and the surplus is deionized water.

Preparation Method:

a. weighing 10 g of carboxymethyl chitosan to be added to 350 g of deionized water to be mixed till being completely dissolved, so as to form liquid A for reservation;

b. weighing 5 g of collagen powder to be added into 200 g of deionized water to be mixed till being completely dissolved, so as to form liquid B for reservation;

c. sequentially weighing 50 g of laurocapram and 2 g of triclosan to be dissolved in 100 g of propanetriol in sequence to be uniformly mixed, so as to form liquid C;

d. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;

e. filling.

### Embodiment II

Components are taken by weight percentage as follows:

By taking 1000 g liquid dressing as reference, 2 percent of carboxymethyl chitosan, 0.4 percent of collagen powder, 12 percent of propanetriol, 0.25 percent of triclosan, 8 percent of laurocapram are taken, and the surplus is deionized water.

Preparation Method:

a. weighing 20 g of carboxymethyl chitosan to be added to 350 g of deionized water to be mixed till being completely dissolved, so as to form liquid A for reservation;

b. weighing 4 g of collagen powder to be added into 200 g of deionized water to be mixed till being completely dissolved, so as to form liquid B for reservation;

c. sequentially weighing 80 g of laurocapram and 2.5 g of triclosan to be dissolved in 120 g of propanetriol in sequence to be uniformly mixed, so as to form liquid C;

d. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;

e. filling.

### Embodiment III

Components are taken by weight percentage as follows:

By taking 1000 g liquid dressing as reference, 4 percent of carboxymethyl chitosan, 0.3 percent of collagen powder, 15 percent of propanetriol, 0.3 percent of triclosan, 10 percent of laurocapram are taken, and the surplus is deionized water.

Preparation Method:

a. weighing 40 g of carboxymethyl chitosan to be added to 350 g of deionized water to be mixed till being completely dissolved, so as to form liquid A for reservation;

b. weighing 3 g of collagen powder to be added into 200 g of deionized water to be mixed till being completely dissolved, so as to form liquid B for reservation;

c. sequentially weighing 100 g of laurocapram and 3 g of triclosan to be dissolved in 150 g of propanetriol in sequence to be uniformly mixed, so as to form liquid C;

d. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;

e. filling.

### Embodiment IV

Components are taken by weight percentage as follows:

By taking 1000 g liquid dressing as reference, 5 percent of carboxymethyl chitosan, 0.1 percent of collagen powder, 15 percent of propanetriol, 0.3 percent of triclosan, 10 percent of laurocapram are taken, and the surplus is deionized water.

Preparation Method:

a. weighing 50 g of carboxymethyl chitosan to be added to 350 g of deionized water to be mixed till being completely dissolved, so as to form liquid A for reservation;

b. weighing 1 g of collagen powder to be added into 200 g of deionized water to be mixed till being completely dissolved, so as to form liquid B for reservation;

c. sequentially weighing 100 g of laurocapram and 3 g of triclosan to be dissolved in 150 g of propanetriol in sequence to be uniformly mixed, so as to form liquid C;

d. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;

e. filling.

### Embodiment V

Components are taken by weight percentage as follows:

By taking 1000 g liquid dressing as reference, 2 percent of carboxymethyl chitosan, 0.4 percent of collagen powder, 12 percent of sorbierite, 0.5 percent of R-polysaccharide and 8 percent of menthol are taken, and the surplus is deionized water.

Preparation Method:

a. weighing 20 g of carboxymethyl chitosan to be added to 350 g of deionized water to be mixed till being completely dissolved, so as to form liquid A for reservation;

b. weighing 4 g of collagen powder to be added into 200 g of deionized water to be mixed till being completely dissolved, so as to form liquid B for reservation;

c. weighing 5 g of R-polysaccharide to be completely dissolved in 100 g of deionized water, so as to form liquid C;

d. sequentially weighing 80 g of menthol and 5 g of R-polysaccharide to be dissolved in 120 g of sorbierite in sequence to be uniformly mixed, so as to form liquid C;

e. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;

f. filling.

### Embodiment VI

Components are taken by weight percentage as follows:

By taking 1000 g liquid dressing as reference, 4 percent of carboxymethyl chitosan, 0.3 percent of collagen powder, 15 percent of carbowax4000, 2 percent of R-polysaccharide and 3 percent of diethylene glycol-ethyl ether are taken, and the surplus is deionized water.

Preparation Method

a. weighing 40 g of carboxymethyl chitosan to be added to 350 g of deionized water to be mixed till being completely dissolved, so as to form liquid A for reservation;

b. weighing 3 g of collagen powder to be added into 200 g of deionized water to be mixed till being completely dissolved, so as to form liquid B for reservation;

c. sequentially weighing 30 g of diethylene glycol-ethyl ether and 20 g of R-polysaccharide to be dissolved in 150 g of carbowax4000 in sequence to be uniformly mixed, so as to form liquid C;

d. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;

e. filling.

### Experimental Example I Product Performance Parameter Detection

After the detection performed on performance parameters of the liquid dressing, products of embodiments 1 to 6 all meet the requirements as follows:

1. Property: light yellow, transparent liquid, uniform.

2. pH: a small amount of samples of embodiments 1 to 6 are taken, and the pH value is measured to be 6 to 8 through a pH meter.

### Experimental Example II Wound Liquid Dressing Sample Bacteriostasis Effect

Testing is performed on the liquid dressing according to cylinder-plate method at Section 1, Chapter 18 of Inspection Manual of Medical Microbiology, the result shows that the products of the invention have obvious bacteriostatic circles, and have obvious inhibitory action to the growth of aureus staphylococcus, coliform bacteria, candida albicans and bacillus pyocyaneus, and the detection results refer to Table I.

TABLE I
Diameters of bacteriostatic circle of each experimental group (mm)

| Diameter of | Aureus | Coliform | Candida | Bacillus |
|---|---|---|---|---|
| bacteriostasis | staphylococcus | bacteria | albicans | pyocyaneus |
| Embodiment I | 18.2 ± 1.2 | 16.2 ± 1.3 | 21.1 ± 1.4 | 11.1 ± 1.5 |
| Embodiment II | 17.5 ± 1.7 | 15.1 ± 1.7 | 19.7 ± 1.3 | 10.2 ± 1.3 |
| Embodiment III | 19.1 ± 1.4 | 14.2 ± 1.5 | 20.1 ± 1.6 | 9.7 ± 1.6 |
| Embodiment IV | 18.0 ± 1.5 | 15.6 ± 1.1 | 19.1 ± 1.2 | 11.3 ± 1.2 |
| Embodiment V | 17.2 ± 1.2 | 16.5 ± 1.6 | 20.5 ± 1.4 | 10.9 ± 1.5 |
| Embodiment VI | 19.4 ± 1.1 | 15.2 ± 1.3 | 19.3 ± 1.7 | 9.8 ± 1.8 |

### Experimental Example III Bacterial Examination of Liquid Dressing Samples

Testing is performed according to relevant regulations of GB/T14233.2-1993 and methods regulated by GB15980-1995 respectively, to test liquid dressing prepared in embodiments 1 to 6, and the results show that samples are bacteria free.

### Experimental Example IV Skin Irritation Index (PII) Examination of to Liquid Dressing Samples

Testing is performed according to closed sensitization test methods regulated by GB/T16886.10-2000, and the results show that the liquid dressing can not cause skin sensitization reaction. The testing results refer to Table II.

TABLE II
Closed sensitization test

| | Embodiment | Embodiment | Embodiment | Embodiment | Embodiment | Embodiment |
|---|---|---|---|---|---|---|
| | I | II | III | IV | V | VI |
| Skin irritation | 0 | 0 | 0 | 0 | 0 | 0 |
| index | | | | | | |
| Skin sensitization | None | None | None | None | None | None |

### Experimental Example V Biological Compatibility Experiment

Liquid dressing of embodiments 1 to 6 is respectively injected into white rabbit bodies, observation is performed at fixed time, and the body weight, eating, body temperature and motion are normal for continuous 8 days, and then the liquid dressing has good biological compatibility.

### Experimental Example VI Comparison Test of Wound Healing

Objects to be tested: liquid dressing prepared in embodiments 1 to 6, and reference substance is Yunan white drug-powder.

1. Procoagulant Effect

Coagulation time measuring method: 10 mg of combined objects to be tested are respectively taken as follow table, 1 ml of fresh blood is added into the combined objects to be tested, the combined objects to be tested are shaken to be uniform, and blood coagulation times are recorded. The carboxymethyl chitosan liquid dressing has procoagulant effect, compared with normal coagulation time, the coagulation time of the carboxymethyl chitosan liquid dressing is averagely reduced by 21 percent, and the coagulation time of the carboxymethyl chitosan liquid dressing is obviously superior to that of yunan white drug-powder. The results refer to Table III.

TABLE III
Coagulation time measuring results (min)

| Coagulation time | | | | Average | Reduced by |
|---|---|---|---|---|---|
| (min) | 1 | 2 | 3 | value | (%) |
| Normal | 9.37 | 9.15 | 8.77 | 9.1 | 0 |
| Coagulation time | | | | | |
| (min) | | | | | |
| Embodiment I | 7.38 | 7.25 | 7.47 | 7.37 | 19.05 |
| (min) | | | | | |
| Embodiment II | 7.01 | 7.25 | 7.09 | 7.12 | 21.79 |
| (min) | | | | | |
| Embodiment III | 6.98 | 7.32 | 7.14 | 7.15 | 21.47 |
| (min) | | | | | |
| Embodiment IV | 6.85 | 7.25 | 7.17 | 7.09 | 22.09 |
| (min) | | | | | |
| Embodiment V | 6.75 | 7.41 | 7.38 | 7.18 | 21.1 |
| (min) | | | | | |
| Embodiment VI | 7.05 | 7.32 | 7.29 | 7.22 | 20.66 |
| (min) | | | | | |
| Yunan white | 8.77 | 8.29 | 8.79 | 8.62 | 5.31 |
| drug-powder | | | | | |
| (min) | | | | | |

2. Wound Healing Promoting Effect

Experimental animals adopt male Wister rats, and each group comprises 10 animals. The animals are observed after being wounded at 3, 6, 10, 15 and 21d respectively. The back of rat is depilated through 8 percent sodium sulfide liquid, and pentobarbital sodium is given to the rat through abdominal cavity 24 h later, a circular all skin incision with the diameter being 1.0 cm is cut through surgical scissors after anaesthesia is successful, sterilization is performed through 70 percent alcohol solution, dressing provided by embodiments 1 to 6 is respectively applied to the wounded surfaces, and the wounded surfaces are covered through paraffin absorbent gauze, and then are dressed through bandages. Normal saline is provided for the wounded surfaces through the abdominal cavity. The dressing of embodiments 1 to 6 is respectively covered on the wounded surfaces, to be served as experimental groups 1 to 6; comparison groups are processed through 0.2 percent ethacridine solution, then bandaging is performed, and wounded surface healing rates are calculated at all sampling time points.

The calculating method of wounded surface healing rate is as follows: firstly the wounded surface is marked on semitransparent paper, then the semitransparent is taken as a template, hard paper with uniform texture is cut into pieces in identical sizes, then the pieces are weighing, and the weight of the piece indirectly represents the size of the wounded surface.

The wounded healing rate is calculated according to the following formula:

Wounded healing rate(%)=(original wounded surface area−unhealed wounded surface area)/original wounded surface area

Pathological examination is performed to know the wounded area healing quality and to fix the healing time according to the wounded surface healing condition. The experimental results are as Table IV:

TABLE IV
Wound healing rates (%) of all experimental groups

| | | Sampling days | | | | |
|---|---|---|---|---|---|---|
| | | 3 d | 6 d | 10 d | 15 d | 21 d |
| Experimental | Wounded surface | 23.7 ± 1.1 | 35.9 ± 1.4 | 60.4 ± 1.4 | 87.1 ± 1.2 | 96.5 ± 1.1 |
| group I | average healing | | | | | |
| | rate | | | | | |
| Experimental | Wounded surface | 21.2 ± 1.6 | 32.7 ± 1.7 | 67.2 ± 1.5 | 86.5 ± 1.2 | 97.4 ± 1.5 |
| group II | average healing | | | | | |
| | rate | | | | | |
| Experimental | Wounded surface | 26.4 ± 1.3 | 36.4 ± 1.8 | 63.7 ± 1.3 | 85.7 ± 1.2 | 98.2 ± 1.3 |
| group III | average healing | | | | | |
| | rate | | | | | |
| Experimental | Wounded surface | 22.3 ± 1.1 | 39.1 ± 1.6 | 60.4 ± 1.7 | 87.4 ± 1.8 | 95.4 ± 1.4 |
| group IV | average healing | | | | | |
| | rate | | | | | |
| Experimental | Wounded surface | 24.1 ± 1.3 | 34.2 ± 1.7 | 59.4 ± 1.3 | 86.9 ± 1.7 | 98.7 ± 1.2 |
| group V | average healing | | | | | |
| | rate | | | | | |
| Experimental | Wounded surface | 23.7 ± 1.5 | 33.6 ± 1.1 | 61.3 ± 1.5 | 87.5 ± 1.1 | 96.3 ± 1.1 |
| group VI | average healing | | | | | |
| | rate | | | | | |
| Comparison | Wounded surface | 4.5 ± 1.1 | 10.8 ± 1.4 | 30.6 ± 1.4 | 50.7 ± 1.7 | 77.2 ± 1.6 |
| group | average healing | | | | | |
| | rate | | | | | |

From Table IV it can be seen that after the liquid dressing is applied, the wounded surface average healing rate of each sampling point is higher than that of the comparison group, and the wounded surface average healing rate is about 97 percent at the 21st day and is about 20 percent higher than that of the comparison group, so that the wound healing promoting effect of the liquid dressing is excellent.

1. Liquid dressing including chitosan derivative, which is characterized in that the liquid dressing comprises the following components by weight percentage: 1 to 5 percent of carboxymethyl chitosan, 0.1 to 0.5 percent of collagen powder, 10 to 15 percent of moisturizing agent, 0.2 to 2 percent of antimicrobial agent, 2 to 10 percent of transdermal enhancer, and the surplus for deionized water.

2. Liquid dressing including chitosan derivative according to claim 1, which is characterized in that the carboxymethyl chitosan adopts O-site substitution, or N-site substitution, or O-site substitution and N-site substitution simultaneously, the molecular weight is within 500 thousands to 3 millions, and the substitution value is within 0.5 to 3.

3. Liquid dressing including chitosan derivative according to claim 1, which is characterized in that the moisturizing agent adopts propanetriol, sorbierite or carbowax.

4. Liquid dressing including chitosan derivative according to claim 1, which is characterized in that the antimicrobial agent adopts triclosan or R-polysaccharide.

5. Liquid dressing including chitosan derivative according to claim 1, which is characterized in that the transdermal enhancer adopts laurocapram, menthol or diethylene glycol-ethyl ether.

6. Liquid dressing including chitosan derivative according to claim 1, which is characterized in that the liquid dressing comprises the following components by weight percentage: 1 to 5 percent of carboxymethyl chitosan, 0.1 to 0.5 percent of collagen powder, 10 to 15 percent of propanetriol, 0.2 to 0.3 percent of triclosan, 5 to 10 percent of laurocapram, and the surplus for deionized water.

7. The preparation method of liquid dressing including chitosan derivative according to claim 1, which is characterized in that the method comprises the following steps:
a. weighing carboxymethyl chitosan to be added into the deionized water to be mixed till being completely dissolved, so as to form liquid A for reservation;
b. weighing collagen powder to be added into the deionized water to be mixed till being completely dissolved, so as to form liquid B for reservation;
c. sequentially weighing the transdermal enhancer and the antimicrobial agent to be dissolved in the moisturizing agent in sequence to be uniformly mixed, so as to form liquid C;
d. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;
e. filling.

8. The method according to claim 7, which is characterized in that the method comprises following steps:
a. weighing carboxymethyl chitosan to be added into the deionized water to be mixed till being completely dissolved, so as to form liquid A for reservation;
b. weighing collagen powder to be added into the deionized water to be mixed till being completely dissolved, so as to form liquid B for reservation;
c. sequentially weighing the laurocapram and the triclosan to be dissolved in the propanetriol in sequence to be uniformly mixed, so as to form liquid C;
d. sequentially pouring the liquid B and the liquid C into the liquid A to be uniformly mixed, so as to form mixed liquid D, adding residual deionized water into the mixed liquid D, and sufficiently mixing the mixed liquid D till being uniformly mixed;
e. filling.
